(19) United States
(12) Patent Application Publication
(10) Pub. No.: US 2023/0255557 A1
(43) Pub. Date: Aug. 17, 2023

(54) ADJUSTABLE HEADBAND FOR BONE CONDUCTION AUDIOMETRIC TESTING

(71) Applicant: Audiology Incorporated, Arden Hills, MN (US)

(72) Inventors: Robert H. Margolis, Arden Hills, MN (US); Janet C. Margolis, Arden Hills, MN (US)

(21) Appl. No.: 17/673,332

(22) Filed: Feb. 16, 2022

(51) Int. Cl. A61B 5/00; A61B 5/12

(52) U.S. Cl. CPC A61B 5/6803; A61B 5/123; A61B 2503/045; A61B 2503/06

(57) ABSTRACT

An audiometric testing headband includes a strap, an anchor, an adult male sizing point, an adult female sizing point, a child sizing point, and an infant sizing point. The anchor is a first type of fastener on a first end of the strap. The sizing points are on the strap and a second type of fastener that mates with the anchor. The adult male sizing point is on a second end of the strap a first distance from the anchor. The adult female sizing point is a second distance from anchor. The child sizing point is a third distance from the anchor. The infant sizing point is a fourth distance from the anchor. Each distance is between a center of the anchor and a center of each respective sizing point.

### BACKGROUND

This application relates to audiometric testing equipment and, more particularly, to a headband for bone conduction threshold testing.

Bone conduction threshold testing uses an electromechanical vibrator or oscillator that vibrates at various frequencies. The vibrator connects to an audiometer, like an AudioStar Pro or Pello made by Grason-Stadler. The vibrator is placed on a patient's head with a specific coupling force. Coupling force is the pressure the vibrator exerts on the head. The vibrator vibrates against the patient's head at a specific frequency. The patient responds when they hear the vibrations. The lowest intensity at which the patient perceives the frequency 50% of the time is called threshold. Testing is repeated for frequencies between 250 Hz and 4,000 Hz. The audiometer controls the frequency and intensity of the vibrator.

Variations in coupling force decrease the accuracy of bone conduction threshold testing. As coupling force increases, the patient's threshold level decreases. Threshold level variations are larger at lower frequencies than at higher frequencies. The American National Standards Institute (S3.6-2018) requires bone conduction threshold audiometric tests have a coupling force of 5.4 N±0.5 N (500-602 grams) to provide accurate results. This is the coupling force necessary for testing in both the forehead and the mastoid positions.

Headbands are of leather or elastic. One previous elastic headband had three closures. Each closure was spaced to approximately fit an adult, a child, and an infant, respectively. The elastic that made up the headband is no longer manufactured.

Another headband is the P-3333 steel headband manufactured by Radioear. The steel headband is designed for mastoid placement and has been used for forehead placement. The device produces more force in the forehead position than in the mastoid position because the head's anterior-posterior distance is greater than its lateral distance. The headband produces forces that are greater than the specified force in the ANSI standard.

### SUMMARY

A headband for forehead-positioned and mastoid-positioned bone conduction audiometric testing in patients with different head sizes, the headband includes a strap, an anchor, an adult male sizing point, an adult female sizing point, a child sizing point, and an infant sizing point. The anchor is on a first end of the strap. The anchor is a first type of fastener. The adult male sizing point is on a second end of the strap a first distance from the anchor. The first distance is between a center of the anchor and a center of the adult male sizing point. The adult female sizing point is on the strap and a second distance from the anchor that is less than the first distance. The second distance is from the center of the anchor and a center of the adult female sizing point. The child sizing point is on the strap and a third distance from the anchor that is less than the second distance. The third distance is from the center of the anchor and a center of the child sizing point. The infant sizing point is on the strap and a fourth distance from the anchor that is less than the third distance. The third distance is between the center of the anchor and a center of the adult female sizing point. The adult male sizing point, the adult female sizing point, the child sizing point, and the infant sizing point are each a second type of fastener that mates with the first type of fastener.

A headband for forehead-positioned and mastoid-positioned bone conduction audiometric testing in patients with different head sizes, the headband includes a strap, an anchor, an adult male sizing point, an adult female sizing point, a child sizing point, and an infant sizing point. The strap is made of one-inch wide double-fold elastic with a stretch factor between 110% and 120%. The anchor is on a first side and a first end of the strap and is a first type of fastener. The adult male sizing point is on a second side and a second end of the strap and is a second type of fastener for mating with the first type of fastener. The adult female sizing point is on the second side of the strap between the first end and the adult male sizing point and is the second type of fastener. The child sizing point is on the second side of the strap between the first end and the adult female sizing point and is the second type of fastener. The infant sizing point is on the second side of the strap between the first end and the child sizing point and is the second type of fastener.

An audiometric testing system includes a headband for bone conduction audiometric testing in patients with different head sizes and an audiometric vibrator. The headband holds the vibrator adjacent to a head of a patient. The headband includes a strap, an anchor, an adult male sizing point, an adult female sizing point, a child sizing point, and an infant sizing point. The anchor is on a first end of the strap. The anchor is a first type of fastener. The adult male sizing point is on a second end of the strap. A center of the adult male sizing point is a first distance from a center of the anchor. The adult male sizing point is a second type of fastener for mating with the first type of fastener. The adult female sizing point is on the strap and is a second distance from the anchor. A center of the adult female sizing point is a second distance from the center of the anchor. The second distance is less than the first distance. The adult female sizing point is the second type of fastener. The child sizing point is on the strap and is a third distance from the anchor. A center of the child sizing point is a third distance from the center of the anchor. The third distance is less than the second distance. The child sizing point is the second type of fastener. The infant sizing point is on the strap and is a fourth distance from the anchor. A center of the infant sizing point is a fourth distance from the center of the anchor. The fourth distance is less than the third distance. The infant sizing point is the second type of fastener.

An audiometric testing system includes a headband for bone conduction audiometric testing in patients with different head sizes and a bone conduction audiometric vibrator. The headband holds the vibrator adjacent to a head of a patient. The headband includes a strap, an anchor, an adult male sizing point, an adult female sizing point, a child sizing point, an infant sizing point, and a newborn sizing point. The anchor is on a first end of the strap. The strap is made of one-inch wide double-fold elastic with a stretch factor between 110% and 120%. The anchor is a first type of fastener. The adult male sizing point is on a second end of the strap. A center of the adult male sizing point is a first distance from a center of the anchor. The adult male sizing point is a second type of fastener for mating with the first type of fastener. The adult male sizing point connects to the anchor to form an adult male wearable loop with a circumference between 16.375 inches (41.59 cm) and 16.625 inches (42.23 cm). The adult male wearable loop holds the audiometric vibrator to a head of an adult male patient with a coupling force between 500 grams and 602 grams. The adult female sizing point is on the strap. A center of the adult female sizing point is a second distance from the center of the anchor. The second distance is less than the first distance. The adult female sizing point is the second type of fastener. The adult female sizing point connects to the anchor to form an adult female wearable loop with a circumference between 15.5 inches (39.37 cm) and 15.75 inches (40.00 cm). The adult female wearable loop holds the audiometric vibrator to a head of an adult female patient with a coupling force between 500 grams and 602 grams. The child sizing point is on the strap. A center of the child sizing point is a third distance from the center of the anchor. The third distance is less than the second distance. The child sizing point is the second type of fastener. The child sizing point connects to the anchor to form a child wearable loop with a circumference between 14.25 inches (36.20 cm) and 14.5 inches (36.83 cm). The child wearable loop holds the audiometric vibrator to a head of a child patient with a coupling force between 500 grams and 602 grams. The infant sizing point is on the strap. A center of the infant sizing point is a fourth distance from the center of the anchor. The fourth distance is less than the third distance. The infant sizing point is the second type of fastener. The infant sizing point connects to the anchor to form an infant wearable loop with a circumference between 12.625 inches (32.07 cm) and 12.875 inches (32.70 cm). The infant wearable loop holds the audiometric vibrator to a head of an infant patient with a coupling force between 500 grams and 602 grams. The newborn sizing point is on the strap. A center of the newborn sizing point is a fifth distance from the center of the anchor. The fifth distance is less than the fourth distance. The newborn sizing point is the second type of fastener. The newborn sizing point connects to the anchor to form a newborn wearable loop with a circumference 11 inches (27.94 cm) and 11.5 inches (29.21 cm). The newborn wearable loop holds the audiometric vibrator to a head of a newborn patient with a coupling force between 500 grams and 602 grams.

### BRIEF DESCRIPTION OF THE DRAWINGS

FIG. 1A is a front view of a bone conduction threshold testing system, including a headband and a vibrator, on an adult male patient undergoing bone conduction threshold audiometric testing in a forehead position.

FIG. 1B is a front view of an adult female patient wearing the headband while undergoing bone conduction threshold testing with the vibrator in the forehead position.

FIG. 1C is a side view of a six-year-old child patient wearing the headband while undergoing bone conduction threshold testing with the vibrator in the mastoid position.

FIG. 1D is a front view of a 6-month-old infant patient wearing the headband while undergoing bone conduction threshold testing with the vibrator in the forehead position.

FIG. 1E is a front view of a newborn infant patient wearing the headband while undergoing bone conduction threshold testing with the vibrator in the forehead position.

FIG. 2A is a front view of the headband with one end folded back to expose an anchor.

FIG. 2B is a front view of the headband.

FIG. 3 is a testing setup to determine coupling force between the vibrator and a head of a test head.

FIG. 4 is a graph showing mean forces produced by the headband on the vibrator when placed in either the forehead position or the mastoid position on five artificial heads.

### DETAILED DESCRIPTION

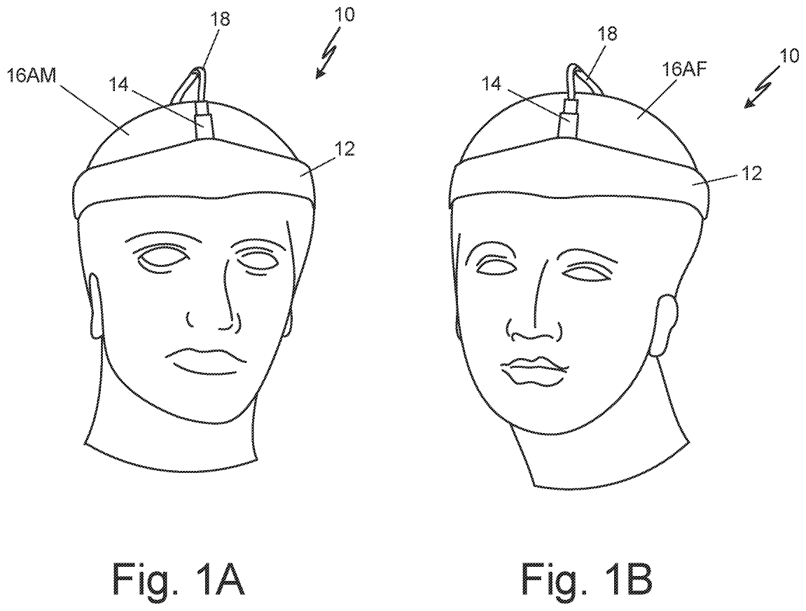

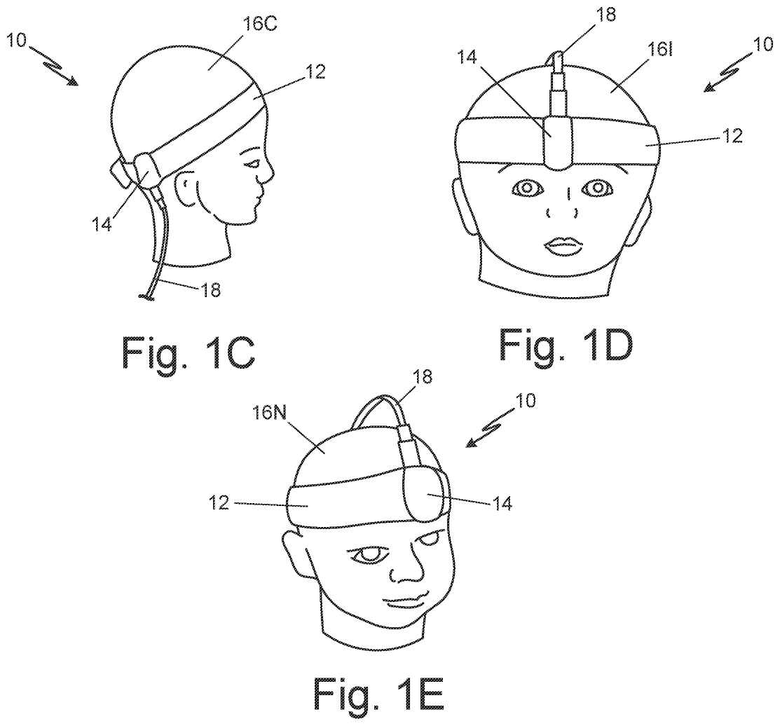

FIG. 1A is a front view of bone conduction threshold testing system 10, including headband 12 and vibrator 14, on adult male patient 16AM undergoing bone conduction threshold audiometric testing in a forehead position. FIG. 1B is a front view of adult female patient 16AF wearing headband 12 while undergoing bone conduction threshold testing with vibrator 14 in the forehead position. FIG. 1C is a side view of six-year-old child patient 16C wearing headband 12 while undergoing bone conduction threshold testing with vibrator 14 in a mastoid position. FIG. 1D is a front view of 6-month-old infant patient 161 wearing headband 12 while undergoing bone conduction threshold testing with vibrator 14 in the forehead position. FIG. 1E is a front view of newborn patient 16N wearing headband 12 while undergoing bone conduction threshold testing with vibrator 14 in the forehead position. FIGS. 1A-1E will be discussed together. FIGS. 1A-1E show audiometric bone conduction threshold testing system 10. Testing system 10 includes headband 12 and bone conduction vibrator 14. FIG. 1A includes patient 16AM, FIG. 1B includes patient 16AF, FIG. 1C includes patient 16C, FIG. 1D includes patient 161, and FIG. 1E includes patient 16N. Testing system 10 also includes cable 18.

Testing system 10 is a bone conduction threshold testing system. Headband 12 encircles a head of a patient. Headband 12 is adjustable to fit patients of varying ages. Vibrator 14 is held against the head of the patient by headband 12. Vibrator 14 can be in a forehead position by resting on a forehead of the patient (as shown in FIG. 1A, FIG. 1B, FIG. 1D, and FIG. 1E). Vibrator 14 can alternatively be in a mastoid position by resting above a mastoid process of the patient (as shown in FIG. 1C). Vibrator 14 connects to a testing booth controlled by an audiologist (not shown). Testing system 10 can be used on a variety of patients. For example, FIG. 1A shows testing system 10 on a head of adult male patient 16AM. Similarly, FIG. 1B shows testing system 10 on a head of adult female patient 16AF, FIG. 1C shows testing system 10 on a head of 6-year-old child patient 16C, FIG. 1D shows testing system 10 on a head of a 6-month-old infant patient 161, and FIG. 1E shows testing system 10 on a head of newborn patient 16N. Cable 18 connects vibrator 18 to an audiometer (not shown). Vibrator 14 and the audiometer can alternatively connect via a wireless connection such as Wi-Fi or Bluetooth.

Testing system 10 is for use in bone conduction threshold testing. Headband 12 holds vibrator 14 to the head of the patient creating a coupling force. Coupling force is the pressure exerted on the head by vibrator 14. In testing system 10, coupling force is dependent on force exerted by headband 12 on vibrator 14. The force exerted by headband 12 depends on the size of headband 12 and a circumference of the head of the patient. Headband 12 is adjustable to create a coupling force between 500 grams and 602 grams on many different patients. Headband 12 can be worn by adult male patient 16AM, adult female patient 16AF, child patient 16C, infant patient 161, and newborn patient 16N. The audiometer controls vibrator 14 through cable 18. Vibrator 14 sends electromechanical vibrations at varying frequencies determined by the audiometer into the head of the patient. The patient communicates their responses to the audiometer using an interface for the audiometer. Threshold is determined by measuring the lowest intensity in decibels the patient perceives the frequency 50% of the time. Bone conduction threshold testing is repeated on the patient with frequencies between 250 Hz to 4,000 Hz.

Testing system 10 accurately measures bone conduction threshold levels. Headband 12 holds vibrator 14 to the heads of patients to create a proper coupling force for bone conduction threshold testing. A proper coupling force is within the recommended range by the American National Standards Institute, which recommends a coupling force of 5.4 N±0.5 N (500-602 grams). ANSI S3.6 (2018). When a bone conduction threshold test is performed with a coupling force outside of the ANSI-recommended range, properties of skin and other soft tissues alter threshold levels. Headband 12 is a single adjustable headband that can be used for adult male patient 16AM, adult female patient 16AF, child patient 16C, infant patient 161 and newborn patient 16N. For each patient, headband 12 holds vibrator 14 to the head of the patient to create a proper coupling force between 500 grams and 602 grams. Headband 12 creates the same force on all portions of the head. So, headband 12 holds vibrator 14 onto the heads of patients with the proper coupling force in both the forehead position and the mastoid position.

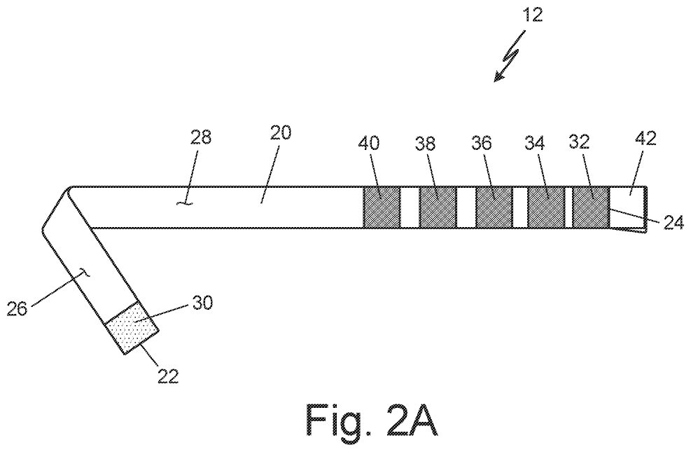

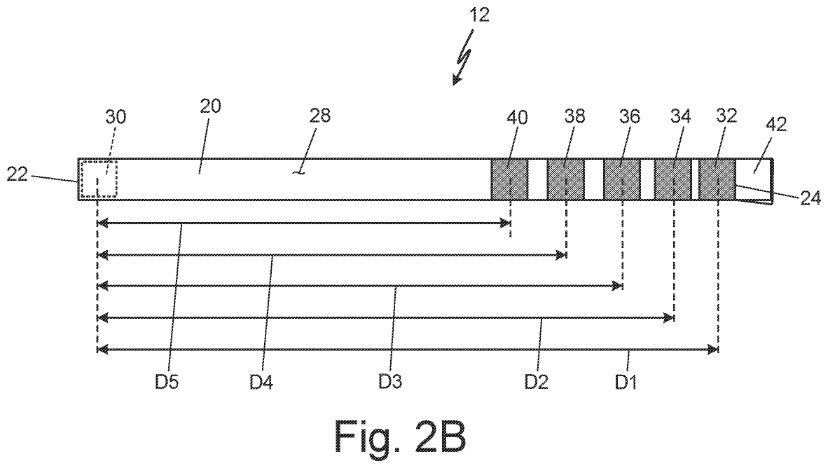

FIG. 2A is a front view of headband 12 with first end 22 folded back to expose anchor 30. FIG. 2B is a front view of headband 12. FIGS. 2A-2B will be discussed together. Headband 12 has strap 20 with first end 22, second end 24, first side 26 (shown in FIG. 2A), and second side 28. Headband 12 also has anchor 30, adult male sizing point 32, adult female sizing point 34, child sizing point 36, infant sizing point 38, newborn sizing point 40, and tab 42. FIG. 2B also includes first distance D1, second distance D2, third distance D3, fourth distance D4, and fifth distance D5.

Headband 12 has strap 20 with first end 22. Second end 24 of strap 20 is opposite first end 22. First side 26 of strap 20 extends from first end 22 to second end 24. Second side 28 of strap 20 is a reverse side of first side 26. Strap 20 of headband 12 is made from one-inch wide double-fold elastic. The elastic can be made from nylon, spandex, polyester, and rubber. Strap 20 has a stretch percentage between 110% and 120%. Strap 20 preferably has a stretch percentage of 114%. Stretch percentage can be calculated with the following process: (1) measure a length of unstretched elastic; (2) holding the elastic at each end of the measured length, stretch the elastic as far as possible without breaking the elastic; (3) measure the length of the stretched elastic; (4) divide the stretched elastic length by the unstretched elastic length and subtract one; and (5) convert the number into a percentage by multiplying by 100.

Anchor 30 connects to first side 26 of strap 20 and is adjacent to first end 22. Adult male sizing point 32 is adjacent to second end 24 on second side 28 of strap 20. Adult female sizing point 34 is on second side 28 of strap 20 between adult male sizing point 32 and first end 22. Child sizing point 36 is on second side 28 of headband 12 between adult female sizing point 34 and first end 22. Infant sizing point 38 is located on a second side 28 of headband 12 between child sizing point 36 and first end 22 of strap 20. Newborn sizing point 40 is on a second side 28 of headband 12 between infant sizing point 38 and second end 24. Adult male sizing point 32, adult female sizing point 34, child sizing point 36, infant sizing point 38, and newborn sizing point 40 are all closer to second end 24 of strap 20 than first end 22. Tab 42 connects to second end 24 of strap 20 adjacent to adult male sizing point 32.

A center of adult male sizing point 32 is located first distance D1 from a center of anchor 30. First distance D1 is between 16.375 inches (41.59 cm) and 16.625 inches (42.23 cm). First distance D1 is preferably 16.5 inches (41.91 cm). A center of adult female sizing point 34 is located second distance D2 from the center of anchor 30. Second distance D2 is between 15.5 inches (39.37 cm) and 15.75 inches (40.00 cm). Second distance D2 is preferably 15.625 inches (39.69 cm). A center of child sizing point 36 is located third distance D3 from the center of anchor 30. Third distance D3 is between 14.25 inches (36.20 cm) and 14.5 inches (36.83 cm). Third distance D3 is preferably 14.375 inches (36.51 cm). A center of infant sizing point 38 is located fourth distance D4 from the center of anchor 30. Fourth distance D4 is between 12.625 inches (32.07 cm) and 12.875 inches (32.70 cm). Fourth distance D4 is preferably 12.75 inches (32.39 cm). A center of newborn sizing point 40 is located fifth distance D5 from the center of anchor 30. Fifth distance D5 is between 11 inches (27.94 cm) and 11.5 inches (29.21 cm). Fifth distance D5 is preferably 11.125 inches (28.26 cm).

Anchor 30 is a first type of fastener. Adult male sizing point 32, adult female sizing point 34, child sizing point 36, infant sizing point 38, and newborn sizing point 40 are each made of a second type of fastener. The second type of fastener mates with the first type of fastener. For example, the first type of fastener could be a male end of a snap, a button, or a piece of hook tape and the second type of fastener could be a female end of a snap, a buttonhole, or a piece of loop tape, respectively. Other closures are also acceptable for anchor 30 and adult male sizing point 32, adult female sizing point 34, child sizing point 36, infant sizing point 38, and newborn sizing point 40.

Anchor 30 connects to adult male sizing point 32 creating an adult male loop. The adult male loop fits an adult male patient, like patient 16 AM (shown in FIG. 1A). The adult male loop has a circumference between 16.375 inches (41.59 cm) and 16.625 inches (42.23 cm). The adult male loop preferably has a circumference of 16.5 inches (41.91 cm). Anchor 30 connects to adult female sizing point 34 creating an adult female loop. The adult female loop fits an adult female patient, like patient 16 AF (shown in FIG. 1B). The adult female loop has a circumference between 15.5 inches (39.37 cm) and 15.75 inches (40.00 cm). The adult female loop preferably has a circumference of 15.625 inches (39.69 cm). Anchor 30 connects to child sizing point 36 creating a child loop. The child loop fits a six-year-old child patient, like patient 16C (shown in FIG. 1C). The child loop has a circumference between 14.25 inches (36.20 cm) and 14.5 inches (36.83 cm). The child loop preferably has a circumference of 14.375 inches (36.51 cm). Anchor 30 connects to infant sizing point 38 creating an infant loop. The infant loop fits a six-month-old infant patient, like patient 161 (shown in FIG. 1D). The infant loop has a circumference between 12.625 inches (32.07 cm) and 12.875 inches (32.70 cm). The infant loop preferably has a circumference of 12.75 inches (32.39 cm). Anchor 30 connects to newborn sizing point 40 creating a newborn loop. The newborn loop fits a newborn patient, like patient 16N (shown in FIG. 1E). The newborn loop has a circumference between 11 inches (27.94 cm) and 11.5 inches (29.21 cm). The newborn loop preferably has a circumference of 11.125 inches (28.26 cm). Tab 42 allows for easy removal of anchor 30 from any of the sizing points.

Using elastic for strap 20 means headband 12 is comfortable and exerts a similar force on all portions of a head. Headband 12 therefore produces the proper coupling force on vibrator 14 (shown in FIGS. 1A-1E) when placed in the forehead position or the mastoid position on a patient.

Adult male sizing point 32, adult female sizing point 34, child sizing point 36, infant sizing point 38, and newborn sizing point 40 make headband 12 adjustable. Depending on the age of a patient, headband 12 can be larger or smaller to create a proper coupling force between 500 grams and 602 grams on a head of a patient wearing headband 12. Placing adult male sizing point 32 distance D1 from anchor 30 on strap 20 means adult male loop creates a coupling force between 500 grams and 602 grams when worn on adult male patient 16AM (shown in FIG. 1A). Placing adult female sizing point 34 distance D2 from anchor 30 on strap 20 means adult female loop creates a coupling force between 500 grams and 602 grams when worn on adult female patient 16FM (shown in FIG. 1B). Placing child sizing point 36 distance D3 from anchor 30 on strap 20 means child loop creates a coupling force between 500 grams and 602 grams when worn on child patient 16C (shown in FIG. 1C). Placing infant sizing point 38 distance D4 from anchor 30 on strap 20 means infant loop creates a coupling force between 500 grams and 602 grams when worn on infant patient 161 (shown in FIG. 1D). Placing newborn sizing point 40 distance D5 from anchor 30 on strap 20 means newborn loop creates a coupling force between 500 grams and 602 grams when worn on newborn patient 16N (shown in FIG. 1E). Including multiple sizing points on strap 20 means headband 12 can be used for accurate threshold testing on patients with different ages and different head sizes.

Adult male sizing point 32, adult female sizing point 34, child sizing point 36, infant sizing point 38, and newborn sizing point 40 are each made of a fastener that complements a fastener used for anchor 30. Using fasteners allows for discrete placement of anchor 30 with each sizing point. Discrete placement increases the repeatability of loop sizing and therefore coupling force created by each loop. This decreases user error. A repeatable coupling force, especially when the repeatable coupling force is a proper coupling force, increases the accuracy of bone conduction threshold testing.

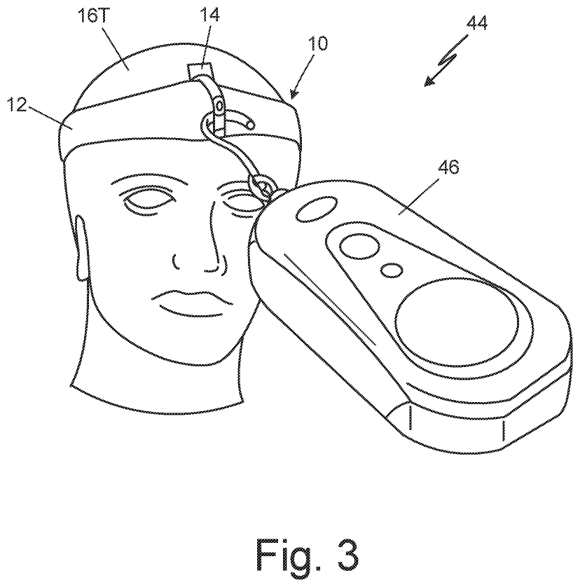

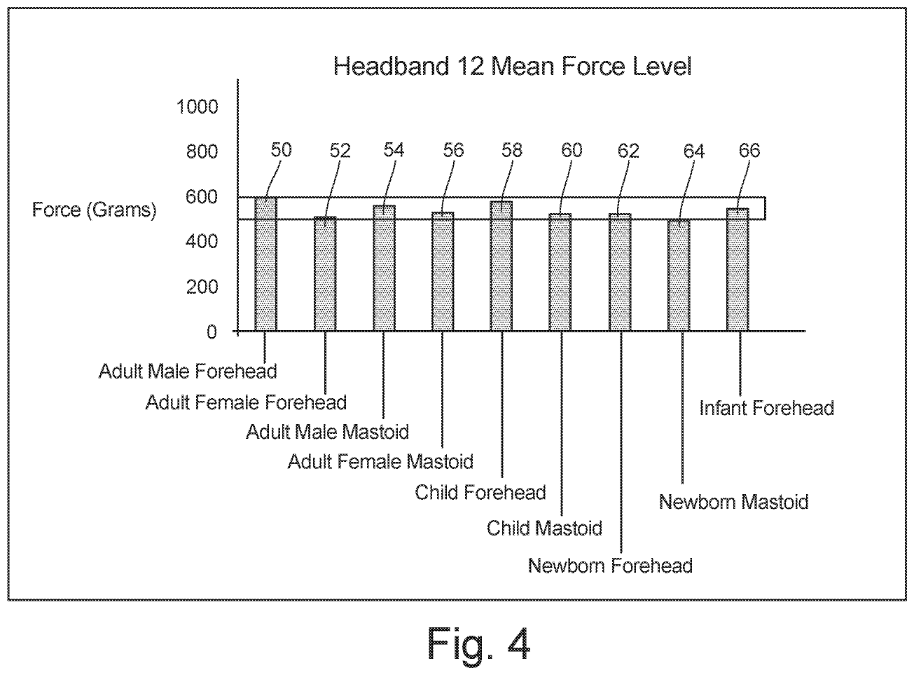

FIG. 3 shows testing setup 44 being used to determine the coupling force between vibrator 14 and a head of test head 16T. FIG. 4 is a graph showing mean forces produced by headband 12 on vibrator 14 when placed in either the forehead position or the mastoid position on five artificial test heads. FIGS. 3-4 will be discussed together. FIG. 3 includes testing setup 44 with bone conduction threshold testing system 10 on a head of test head 16T and scale 46. Testing system 10 includes headband 12 and vibrator 14. FIG. 4 is a bar graph. The Y-axis of the graph shows force measured in grams. The Y-axis starts at 0 grams and ends at 1,000 grams. The x-axis shows different test heads who underwent coupling force testing using testing setup 44. The bar graph includes bar 50, bar 52, bar 54, bar 56, bar 58, bar 60, bar 62, bar 64, and bar 66. Each bar represents the mean of 5 measurements using testing setup 44. FIG. 4 includes a box around a range showing proper coupling forces between 500 grams and 602 grams.

Testing setup 44 has testing system 10 on a head of test head 16T. Headband 12 encircles a head of test head 16T. Vibrator 14 is held against the head of test head 16T by headband 12 with a coupling force. Vibrator 14 can be in the forehead position (shown in FIG. 3) or in the mastoid position (shown in FIG. 1C). Scale 46 connects to vibrator 14 but not headband 12. In testing setup 44, scale 46 connects to a loop on vibrator 14. Headband 12 is threaded through the loop. Test head 16T could be an adult male (like patient 16AM in FIG. 1A), an adult female (like patient 16AF in FIG. 1B), a child (like patient 16C in FIG. 1C), an infant (like patient 161 in FIG. 1D), or a newborn (like patient 16N in FIG. 1E).

Testing system 10 is for bone conduction threshold testing as described in relation to FIGS. 1A-1E. Headband 12 holds vibrator 14 to a head of a patient with a coupling force. Testing setup 44 uses scale 46 to measure the coupling force. The coupling force is the force headband 12 exerts on vibrator 14 to keep vibrator 14 against the head of test head 16T. Scale 46 measures the force headband 12 exerts on vibrator 14 by pulling vibrator 14 away from the head of test head 16T. The force headband 12 exerts on vibrator 14 as scale 46 pulls vibrator 14 from the head of test head 16T is coupling force when vibrator 14 slightly separates from the head of test head 16T.

FIG. 4 shows the results of performing tests using test setup 44 on five different artificial heads. The five artificial heads have head circumferences within the normal range for each population (e.g., adult males, adult females, six-year-old children, six-month-old infants, and newborns). Bar 50 is nearest the y-axis and represents a mean of five coupling force tests where test head 16T is an adult male and vibrator 14 was in the forehead position. Bar 52 is to the right of bar 50. Bar 52 represents a mean of five coupling force measurements where test head 16T is an adult female and vibrator 14 is in the forehead position. Bar 54 is to the right of bar 52. Bar 54 represents a mean of five coupling force measurements where test head 16T is an adult male and vibrator 14 is in the mastoid position. Bar 56 is to the right of bar 54. Bar 56 represents a mean of five coupling force measurements where test head 16T is an adult female and vibrator 14 is in the mastoid position. Bar 58 is to the right of bar 56. Bar 58 represents a mean of five coupling force measurements where test head 16T is a six-year-old child and vibrator 14 is in the forehead position. Bar 60 is to the right of bar 58. Bar 60 represents a mean of five coupling force measurements using testing setup 44 where test head 16T is a child and vibrator 14 is in the mastoid position. Bar 62 is to the right of bar 60. Bar 62 represents a mean of five coupling force measurements using testing setup 44 where test head 16T is a newborn and vibrator 14 is in the forehead position. Bar 64 is to the right of bar 62. Bar 64 represents a mean of five coupling force measurements using testing setup 44 where test head 16T is a newborn and vibrator 14 is in the mastoid position. Bar 66 is to the right of bar 64. Bar 66 represents a mean of five coupling force measurements using testing setup 44 with test head 16T is a six-month-old infant and vibrator 14 is in the forehead position. Each bar terminates in the box representing the ANSI-recommended coupling force between 500 grams and 602 grams.

Testing setup 44 is a method to measure a force produced by headband 12 on vibrator 14 and therefore coupling force. FIG. 4 shows that average force levels for each test head 16T are in the ANSI-specified range between 500 grams and 602 grams. This is true when vibrator 14 is in the forehead position or the mastoid position. These measurements indicate that headband 12 reliably produces a proper coupling force, as recommended by ANSI, when headband 12 holds vibrator 14 to heads of five types of patients. Testing with a proper coupling force increases the accuracy of bone conduction threshold testing.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A headband for forehead-positioned and mastoid-positioned bone conduction audiometric testing in patients with different head sizes, the headband comprising:
a strap;
an anchor positioned on a first end of the strap, wherein the anchor is a first type of fastener;
an adult male sizing point on a second end of the strap and a first distance from the anchor, wherein the first distance is between a center of the anchor and a center of the adult male sizing point;
an adult female sizing point on the strap and a second distance from the anchor, wherein the second distance is between the center of the anchor and a center of the adult female sizing point, and wherein the second distance is less than the first distance;
a child sizing point on the strap and a third distance from the anchor, wherein the third distance is between the center of the anchor and a center of the child sizing point, and wherein the third distance is less than the second distance; and
an infant sizing point on the strap and a fourth distance from the anchor, wherein the fourth distance is between the center of the anchor and a center of the infant sizing point, and wherein the fourth distance is less than the third distance;
wherein the adult male sizing point, the adult female sizing point, the child sizing point, and the infant sizing point are each a second type of fastener that mates with the first type of fastener.

2. The headband of claim 1, and further comprising:
a newborn sizing point on the strap and a fifth distance from the anchor, wherein the second distance is between the center of the anchor and a center of the newborn sizing point, wherein the fifth distance is less than the fourth distance, and wherein the newborn sizing point is the second type of fastener.

3. The headband of claim 2, wherein the first distance is between 16.375 inches (41.59 cm) and 16.625 inches (42.23 cm); wherein the second distance is between 15.5 inches (39.37 cm) and 15.75 inches (40.00 cm); wherein the third distance is between 14.25 inches (36.20 cm) and 14.5 inches (36.83 cm); wherein the fourth distance is between 12.625 inches (32.07 cm) and 12.875 inches (32.70 cm); and wherein the fifth distance is between 11 inches (27.94 cm) and 11.5 inches (29.21 cm).

4. The headband of claim 3, wherein the first distance is 16.5 inches (41.91 cm), the second distance is 15.625 inches (39.69 cm), the third distance is 14.375 inches (36.51 cm), the fourth distance is 12.75 inches (32.39 cm), and the fifth distance is 11.125 inches (28.26 cm).

5. The headband of claim 2, wherein the anchor is a first piece of hook-and-loop tape and the adult male sizing point, the adult female sizing point, the child sizing point, the infant sizing point, and the newborn sizing point are each a piece of hook-and-loop tape that mates with the first piece of hook-and-loop tape.

6. The headband of claim 5, wherein the anchor is on a first side of the strap and the adult male sizing point, the adult female sizing point, the child sizing point, the infant sizing point, and the newborn sizing point are located on a second side of the strap, so each sizing point creates a continuous loop when that sizing point connects to the anchor.

7. The headband of claim 1, wherein the strap is comprised of elastic material selected from the group consisting of nylon, spandex, polyester, rubber, and combinations thereof.

8. A headband for forehead-positioned and mastoid-positioned bone conduction audiometric testing in patients with different head sizes, the headband comprising:
a strap made of one-inch wide double-fold elastic with a stretch factor between 110% and 120%;
an anchor positioned on a first side and a first end of the strap, wherein the anchor is a first type of fastener;
an adult male sizing point positioned on a second side and a second end of the strap, wherein the adult male sizing point is a second type of fastener for mating with the first type of fastener;
an adult female sizing point positioned on the second side of the strap between the first end and the adult male sizing point, wherein the adult female sizing point is the second type of fastener;
a child sizing point positioned on the second side of the strap between the first end and the adult female sizing point, wherein the child sizing point is the second type of fastener; and
an infant sizing point positioned on the second side of the strap between the first end and the child sizing point, wherein the infant sizing point is the second type of fastener.

9. The headband of claim 8, and further comprising:
a newborn sizing point positioned on the second side of the strap between the first end and the infant sizing point, wherein the infant sizing point is the second type of fastener.

10. The headband of claim 9, wherein the elastic has a stretch factor between 110% and 120%, and wherein the elastic has a stretch factor of 114%.

11. The headband of claim 10, wherein the elastic is comprised of a material selected from the group consisting of nylon, spandex, polyester, rubber, and combinations thereof.

12. The headband of claim 10, wherein the anchor connects to:
the adult male sizing point creating a wearable loop that holds an audiometric bone conduction vibrator to a head of an adult male patient with a coupling force between 500 grams and 602 grams;
the adult female sizing point creating a wearable loop that holds an audiometric bone conduction vibrator to a head of an adult female patient with a coupling force between 500 grams and 602 grams;
the child sizing point creating a wearable loop that holds an audiometric bone conduction vibrator to a head of a child patient with a coupling force between 500 grams and 602 grams;
the infant sizing point creating a wearable loop that holds an audiometric bone conduction vibrator to a head of an infant patient with a coupling force between 500 grams and 602 grams; or
the infant sizing point creating a wearable loop that holds an audiometric bone conduction vibrator to a head of an infant patient with a coupling force between 500 grams and 602 grams.

13. The headband of claim 9, wherein the first type of fastener is hook tape and the second type of fastener is loop tape.

14. An audiometric testing system comprising:
a headband for bone conduction audiometric testing in patients with different head sizes, the headband comprising:
a strap;
an anchor positioned on a first end of the strap, wherein the anchor is a first type of fastener;
an adult male sizing point on a second end of the strap, wherein a center of the adult male sizing point is a first distance from a center of the anchor, and wherein the adult male sizing point is a second type of fastener for mating with the first type of fastener;
an adult female sizing point on the strap, wherein a center of the adult female sizing point is a second distance from the center of the anchor, wherein the second distance is less than the first distance, and wherein the adult female sizing point is the second type of fastener;
a child sizing point on the strap, wherein a center of the child sizing point is a third distance from the center of the anchor, wherein the third distance is less than the second distance, and wherein the child sizing point is the second type of fastener; and
an infant sizing point on the strap, wherein a center of the infant sizing point is a fourth distance from the center of the anchor, wherein the fourth distance is less than the third distance, and wherein the infant sizing point is the second type of fastener; and
an audiometric vibrator held adjacent to a head of a patient by the headband.

15. The audiometric testing system of claim 14, wherein the headband further comprises:
a newborn sizing point on the strap, wherein a center of the newborn sizing point is a fifth distance from the center of the anchor, wherein the fifth distance is less than the fourth distance, and wherein the newborn sizing point is the second type of fastener.

16. The audiometric testing system of claim 15, wherein the first distance is between 16.375 inches (41.59 cm) and 16.625 inches (42.23 cm); wherein the second distance is between 15.5 inches (39.37 cm) and 15.75 inches (40.00 cm); wherein the third distance is between 14.25 inches (36.20 cm) and 14.5 inches (36.83 cm); wherein the fourth distance is between 12.625 inches (32.07 cm) and 12.875 inches (32.70 cm); and wherein the fifth distance is between 11 inches (27.94 cm) and 11.5 inches (29.21 cm).

17. The audiometric testing system of claim 16, wherein the first distance is 16.5 inches (41.91 cm), the second distance is 15.625 inches (39.69 cm), the third distance is 14.375 inches (36.51 cm), the fourth distance is 12.75 inches (32.39 cm), and the fifth distance is 11.125 inches (28.26 cm).

18. The audiometric testing system of claim 16, wherein the anchor connects to:
the adult male sizing point creating an adult male wearable loop with a circumference between 16.375 inches (41.59 cm) and 16.625 inches (42.23 cm);
the adult female sizing point creating an adult female wearable loop with a circumference between 15.5 inches (39.37 cm) and 15.75 inches (40.00 cm);
the child sizing point creating a child wearable loop with a circumference between 14.25 inches (36.20 cm) and 14.5 inches (36.83 cm);
the infant sizing point creating an infant wearable loop with a circumference between 12.625 inches (32.07 cm) and 12.875 inches (32.70 cm); or
the newborn sizing point creating a newborn wearable loop with a circumference 11 inches (27.94 cm) and 11.5 inches (29.21 cm).

19. The audiometric testing system of claim 18, wherein the audiometric vibrator is a bone-conduction vibrator.

20. The audiometric testing system of claim 19, wherein the adult male wearable loop holds the audiometric vibrator to a head of an adult male patient with a coupling force between 500 grams and 602 grams, the adult female wearable loop holds the audiometric vibrator to a head of an adult female patient with a coupling force between 500 grams and 602 grams, the child wearable loop holds the audiometric vibrator to a head of a child patient with a coupling force between 500 grams and 602 grams, the infant wearable loop holds the audiometric vibrator to a head of an infant patient with a coupling force between 500 grams and 602 grams, the newborn wearable loop holds the audiometric vibrator to a head of a newborn patient with a coupling force between 500 grams and 602 grams.

21. An audiometric testing system comprising:
a headband for bone conduction audiometric testing in patients with different head sizes, the headband comprising:
a strap made of one-inch wide double-fold elastic with a stretch factor between 110% and 120%;
an anchor positioned on a first end of the strap, wherein the anchor is a first type of fastener;
an adult male sizing point on a second end of the strap, wherein a center of the adult male sizing point is a first distance from a center of the anchor, wherein the adult male sizing point is a second type of fastener for mating with the first type of fastener, and wherein the adult male sizing point connects to the anchor to form an adult male wearable loop with a circumference between 16.375 inches (41.59 cm) and 16.625 inches (42.23 cm);
an adult female sizing point on the strap, wherein a center of the adult female sizing point is a second distance from the center of the anchor, wherein the second distance is less than the first distance, wherein the adult female sizing point is the second type of fastener, and wherein the adult female sizing point connects to the anchor to form an adult female wearable loop with a circumference between 15.5 inches (39.37 cm) and 15.75 inches (40.00 cm);
a child sizing point on the strap, wherein a center of the child sizing point is a third distance from the center of the anchor, wherein the third distance is less than the second distance, wherein the child sizing point is the second type of fastener, and wherein the child sizing point connects to the anchor to form a child wearable loop with a circumference between 14.25 inches (36.20 cm) and 14.5 inches (36.83 cm);
an infant sizing point on the strap, wherein a center of the infant sizing point is a fourth distance from the center of the anchor, wherein the fourth distance is less than the third distance, wherein the infant sizing point is the second type of fastener, and wherein the infant sizing point connects to the anchor to form an infant wearable loop with a circumference between 12.625 inches (32.07 cm) and 12.875 inches (32.70 cm); and
a newborn sizing point on the strap and a fifth distance from the anchor, wherein a center of the newborn sizing point is a fifth distance from the center of the anchor, wherein the fifth distance is less than the fourth distance, wherein the newborn sizing point is the second type of fastener, and wherein the newborn sizing point connects to the anchor to form a newborn wearable loop with a circumference 11 inches (27.94 cm) and 11.5 inches (29.21 cm); and
a bone conduction audiometric vibrator held adjacent to a head of a patient by the headband;
wherein the adult male wearable loop holds the audiometric vibrator to a head of an adult male patient with a coupling force between 500 grams and 602 grams;
wherein the adult female wearable loop holds the audiometric vibrator to a head of an adult female patient with a coupling force between 500 grams and 602 grams;
wherein the child wearable loop holds the audiometric vibrator to a head of a child patient with a coupling force between 500 grams and 602 grams;
wherein the infant wearable loop holds the audiometric vibrator to a head of an infant patient with a coupling force between 500 grams and 602 grams; and
wherein the newborn wearable loop holds the audiometric vibrator to a head of a newborn patient with a coupling force between 500 grams and 602 grams.
